(19) United States
(12) Patent Application Publication
(10) Pub. No.: US 2017/0274052 A1
(43) Pub. Date: Sep. 28, 2017

(54) METHOD AND APPARATUS FOR HARVESTING, CREATING AND IMPLANTING A FIBRIN CLOT BIOMATERIAL

(71) Applicant: PSC Cosmetics Ltd., Tsuen Wan (CN)

(72) Inventor: Javin C. Pierce, Waterbury, VT (US)

(21) Appl. No.: 15/452,301

(22) Filed: Mar. 7, 2017

Related U.S. Application Data
(63) Continuation of application No. 14/160,093, filed on Jan. 21, 2014, now abandoned.
(60) Provisional application No. 61/923,035, filed on Jan. 2, 2014.

(51) Int. Cl. A61K 38/36; A61K 35/28

(52) U.S. Cl. CPC A61K 38/36; A61K 35/28; A61K 2300/00

(57) ABSTRACT

A composite biomaterial comprising at least one blood component and adipose-derived stem cells wherein the at least one blood component is bonded to the adipose-derived stem cells as the at least one blood component coagulates.

### REFERENCE TO PENDING PRIOR PATENT APPLICATION

This patent application claims benefit of pending prior U.S. Provisional Patent Application Ser. No. 61/923,035, filed Jan. 2, 2014 by Javin C. Pierce for METHOD AND APPARATUS FOR HARVESTING CREATION AND IMPLANTATION OF A FIBRIN CLOT BIOMATERIAL (Attorney's Docket No. PIERCE-16 PROV), which patent application is hereby incorporated herein by reference.

### FIELD OF THE INVENTION

This invention relates to surgical methods and apparatus in general, and more particularly to novel methods and applications for creating bodily-derived compositions and/or implanting bodily-derived compositions into the body during a medical procedure.

### BACKGROUND OF THE INVENTION

In many situations, it may be necessary or desirable to deploy an implant into the body. In some cases, the implant may be relatively stiff or rigid (e.g., an orthopedic implant). In other cases, the implant may be relatively soft and pliable (e.g., a cosmetic implant).

In some cases, the implant may not integrate well with the adjacent tissue. This can be particularly problematic in cosmetic applications, where implant integration with surrounding tissue can be critical for proper tissue support and proper tissue appearance.

### SUMMARY OF THE INVENTION

The present invention comprises the provision and use of a novel bodily-derived composition.

In one preferred form of the present invention, there is provided a composite biomaterial comprising at least one blood component and adipose-derived stem cells wherein the at least one blood component is bonded to the adipose-derived stem cells as the at least one blood component coagulates.

In another preferred form of the present invention, there is provided a method for making a composite biomaterial comprising:

extracting at least one blood component from a body;

extracting adipose-derived stem cells from a body;

placing the at least one blood component and adipose-derived stem cells in a vessel; and

bonding the at least one blood component to the adipose-derived stem cells as the at least one blood component coagulates.

In another preferred form of the present invention, there is provided a method for treating a patient, the method comprising:

extracting at least one blood component from a body;

extracting adipose-derived stem cells from a body;

placing the at least one blood component and adipose-derived stem cells in a vessel;

activating at least one blood component so that at least one blood component binds to the adipose-derived stem cells as the at least one blood component coagulates; and deploying the composite biomaterial into a human body.

In another preferred form of the present invention, there is provided apparatus for making a composite biomaterial, the apparatus comprising:
a vessel for holding liquids;
a lid for selectively closing off the vessel; and
a precipitator.

In another preferred form of the present invention, there is provided a method for treating a patient, the method comprising:

extracting at least one blood component from a body;

extracting adipose-derived stem cells from a body;

placing the at least one blood component in a vessel;

clotting the at least one blood component in a vessel;

mixing the adipose-derived stem cells with the at least one clotted blood component to form a composite biomaterial; and deploying the composite biomaterial into a human body.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic perspective view showing the present invention;

FIG. 2 is an exploded view of selected components of the present invention;

FIG. 3 is a partially-exploded view of selected components of the present invention;

FIGS. 4A and 4B are side views of selected components of the present invention shown in use;

FIG. 5A is a top view of selected components of the present invention;

FIG. 5B is a top view of an alternative embodiment of selected components of the present invention;

FIG. 5C is a top view of another alternative embodiment of selected components of the present invention;

FIG. 6 is a cross-sectional view of selected components of the present invention;

FIG. 7 is another cross-sectional view of selected components of the present invention;

FIG. 8 is still another cross-sectional view of selected components of the present invention;

FIG. 9A is a schematic cross-sectional view of the present invention;

FIG. 9B shows a clot having circumferentially oriented fibers with an indentation therein formed in accordance with the present invention;

FIG. 9C is a close-up view of a the precipitator component of the present invention showing air bubbles formed thereon;

FIG. 9D shows an alternative embodiment of the present invention wherein a portion of a clot formed in accordance with the present invention is being aspirated;

FIG. 9E is another view an alternative embodiment of the present invention wherein a portion of a clot formed in accordance with the present invention is being aspirated;

FIG. 10 is a cross-sectional view of selected components of yet another embodiment of the present invention;

FIG. 11 is a cross-sectional view of selected components of the present invention with the vessel component of the invention rotated 180 degrees with respect to its position shown in FIG. 10;

FIG. 12 is a cross-sectional view of the present invention with its leveling armature deployed;

FIG. 13 is a schematic perspective view of another alternative embodiment of the present invention;

FIG. 14 is a schematic perspective view of an alternative embodiment of the present invention;

FIGS. 15A-15G show selected components of an alternative embodiment of the present invention;

FIG. 16 is a flow chart showing the various steps of a method of using the present invention;

FIG. 17A shows selected components of the present invention;

FIG. 17B shows selected components of the present invention and portions of a clot formed in accordance the present invention;

FIG. 17C shows selected components of the present invention and portions of a clot formed in accordance the present invention;

FIG. 17D shows a cross-section of a clot formed in accordance with the present invention;

FIG. 17E is another view of a clot formed in accordance with the present invention;

FIG. 17F shows another aspect of a clot formed in accordance with the present invention;

FIG. 17G shows yet another aspect of a clot formed in accordance with the present invention;

FIG. 17H shows still another aspect of a clot formed in accordance with the present invention;

FIG. 18 shows a clot formed in accordance with the present invention;

FIG. 18A shows a clot formed in accordance with the present invention being aspirating into a syringe;

FIG. 19 shows selected components of the present invention;

FIG. 20A shows selected components of the present invention;

FIG. 20B shows a composite implant formed in accordance with the present invention;

FIG. 20C shows a composite implant formed in accordance with the present invention being manipulated by a handle;

FIG. 20D shows another view of a composite implant formed in accordance with the present invention being manipulated by a handle;

FIG. 20E shows yet another view of a composite implant formed in accordance with the present invention;

FIG. 20F shows still another view of a composite implant formed in accordance with the present invention;

FIG. 20G shows yet another view of a composite implant formed in accordance with the present invention being manipulated by a handle;

FIG. 21A is a representative diagram of a human breast;

FIG. 21B is a representative diagram of a human breast after an implant formed in accordance with the present invention is deployed subglandularly in said breast; and

FIG. 21C is a representative diagram of a human breast after an implant formed in accordance with the present invention is deployed submuscularly in said breast.

### DETAILED DESCRIPTION OF THE PREFERRED EMBODIMENTS

This invention relates to aesthetic, surgical, and injection methods and apparatus in general, and more particularly, to a novel method and apparatus for forming, harvesting, and implanting autologous blood-derived fibrin clot biomaterials, optionally combining these with autologous or allogenic cells, synthetic implants, injectable fillers, supports, and the like, and depositing these into the body of a mammal during a medical procedure. The invention also relates to apparatus that induces fluidic motion within a container to elicit selectively-variable implant matrixes as tissue fillers and cell delivery scaffolding means, and biomaterials.

It is also an object of the present invention to provide systems in the form of kits and apparatus to facilitate novel procedures pertaining to forming completely autologous fibrin adipose composite biomaterials by harvesting platelets from blood, harvesting subcutaneous adipocytes, optionally purifying and or disassociating these, combining the blood with the fat in a container or tubing, and inducing motion upon the liquid in a vessel that will trigger clotting by fluidic interaction with the interior of a container.

The creation of a hematoma or fibrin clot is an initial and important phase in wound repair. The fibrin clot is formed from platelets circulating in blood and provides a matrix scaffold as well as a chemotactic stimulus to the various cellular elements involved in wound repair. The fibrin clot is typically a naturally-occurring response to an injury to vascularized tissue.

In the past, several studies have demonstrated the role of platelet rich plasma (PRP) in enhancing tissue healing. PRP has been shown to contain growth factors essential to the natural healing process. However, several shortcomings of PRP have been reported. PRP production typically requires a centrifuge, is expensive, time-consuming, and is inconsistent depending on the patient and the preparation method. In addition, most methods involve a chemical activation step that activates any latent growth factors, including TGF-β1. PRP in liquid form diffuses upon injection into the body, prohibiting long term contact with the injured area. Lastly, PRP in gel form lacks a porous architecture and has no counterpart in nature. This PRP form has been associated with poor outcomes compared to negative controls.

The present invention overcomes the shortcomings of PRP by obviating a centrifuge and additives used to prevent and trigger clotting. The present invention can be practiced with minimal equipment and typically within a 10 minute time period.

The aesthetic skin treatment marketplace has adopted centrifuge-derived platelet rich plasma (PRP) forms for a variety of skin wrinkle applications for skin ageing. These are primarily treated by injection, however, the natural scaffolding for filling voids, suturing, or adding bulk with PRP is lacking unless bulking agents such as collagen or cells are added.

Injected PRP has also gained notoriety in aesthetic surgery as filler and to build epidermis. Autologous adipose tissue transfer (or fat transfer) is another popular treatment for facial skin ageing and for aesthetic augmentations such as for breast augmentation.

Examination of the morphology of the fibrin biomaterial was performed and assessments made as to the presence of the desired growth factors. Using rabbit polyclonal antibodies from abcam, immunohistochemistry was performed to assess the presence of growth factors PDGF-ββ, TGF-β1, VEGF, and FGF basic. For each growth factor, positive staining was compared to a negative control.

The results of this study show that growth factors essential to tissue healing are present in a human blood clot produced by the present invention. This indicates that these man-made blood clots will enhance tissue healing at least as well as PRP. The study also indicates that for all growth factors there is a greater staining intensity around the periphery of the blood clot (where the blood made physical contact with the Hula cup) as compared to the center of the clot. In addition, there is a higher intensity of TGF-β and VEGF staining in the clots than PDGF-ββ and FGF-2. The high presence of VEGF is notable as it is associated with vascular ingrowth which is essential for engrafted cell survivability and matrix regeneration. Conversely, the relatively low presence FGF-2 is associated with minimal production of less desirable fibrosis and scar tissue formation.

Positive staining for VEGF, TGF-β1, and PDGF-ββ indicates that clots produced by the present invention have viable tissue-healing properties. The fact that these bioactive components are present shows that these fibrin biomaterials are capable of enhancing healing at least as well as traditional centrifuge derived Platelet Rich Plasma, without the expensive, time-consuming, and inconsistent preparation process associated with PRP. The present invention produces biomaterials that have similar growth factor content to PRP, and are 3D scaffolds that can be made economically, consistently, and in typically less than 15 minutes. The biomaterial of the present invention also possesses potentially superior tissue ingrowth properties due to its open architecture and slow release of growth factors.

The present invention produces an autologous fibrin clot scaffold that is an ideal biomaterial for tissue regeneration and addresses the deficiencies of the art as an autologous fibrin clot can now be formed into virtually any shape, density and a range of fiber sizes and orientations to enable a variety of target types, payloads and implantation methods. Autologous fibrin biomaterial can provide an optimized structural microenvironment for stem cell therapy that is also laden with a physiologically-balanced mixture of growth factors having elution kinetics refined by millions of years of evolution.

Transplantation of adipose tissue-derived stem cells is a treatment in which stem cells are extracted from adipose tissue, concentrated, and re-injected together with injectable fat. Some cosmetic surgery clinics claiming “breast augmentation by transplantation of adipose tissue-derived stem cells” perform breast augmentation together with injection of drugs for fat retention and prevention of implant necrosis. Conventional breast augmentation procedures include fat injection, hyaluronic acid (Sub-Q) injection, and insertion of a bag prosthesis (artificial bag). Breast augmentation by transplantation of adipose tissue-derived stem cells is a well-accepted technique, however, issues with cell survival and structural integration limit its effectiveness. In an embodiment of the present invention, stem cells collected from the patient's own adipose tissues are concentrated and separated from non-viable lipids and cells, and are added to the fibrin biomaterial, and re-injected into the body together with the fat as a natural composite biomaterial. The fibrin biomaterial provides a cohesive physical biodegradable structural matrix that can be molded into implant shapes or, alternately, can be easily injected into target tissues. Fibrin clot is rich in growth factors, and the presence of vascular endothelial growth factors is significant because an adequate blood supply improves tissue graft viability. Because transplanted stem cells differentiate into new adipose tissue or vascular endothelial cells, this procedure has the advantages of a much higher fat graft survival (retention) rate and longer survival compared with common fat injection. Because fibrin-adipose biomaterials can be made from the patient's own blood and adipose tissue without additives, this represents a safer more cost effective treatment with no concern for rejection or drug side effects.

It is also an object of the present invention to trigger and complete a clotting cascade in vitro, using the patient's own blood and stem cells in order to form a fibrin biomaterial that is directly bonded to stem cells to facilitate both implantation and long-term viability of the implanted cells.

A body of evidence is emerging which points to the role of the microenvironment in influencing stem cell fate. For example, Yin reported: “The expression of tendon-specific genes was significantly higher in hTSPCs growing on aligned nanofibers than those on randomly-oriented nanofibers in both normal and osteogenic media.” PRP and previously known autologous biomaterials have not been repeatably formed in vitro with distinct fiber orientations.

Thus there is a need for a new and improved method and apparatus for forming and harvesting fibrin-adipose biomaterials and for the implantation thereof.

The present invention provides a new and improved method and apparatus for harvesting a fibrin clot and creating and implanting fibrin-adipose biomaterial into the body during a clinical or surgical procedure.

### Creation of Varied Density and Structural Morphology of Three-Dimensional Platelet Rich Fibrin Scaffolds

The invention comprises a method and apparatus for formation of a diverse range of biomaterials and extracellular matrix (ECM) from whole blood and cells without additives, centrifugation or filtration. A unique aspect of the invention described herein is that it enables the user to selectively create fibrin biomaterials possessing a diverse range of properties that can be more conducive to repair and regeneration. Specifically, the fibrin scaffolds promote tissue regeneration and repair, can be made with specific microenvironments (fibril size, orientation and spacing), and release concentrated growth factors over a longer period of time than chemically-activated PRP. The present invention comprises a (preferably) closed container to hold blood and fat, application of motion to the container, and a means to trigger the clotting cascade by turbulence, mixing and/or contact with air (such as with roughened glass). It will be evident to those skilled in the art that impellers, pumps or alternate means to induce fluid motion within a container can be utilized to practice the present invention. The present invention enables the user to optimize the fiber orientation of the biomaterial in strips, laminates, and tendon-like structures that match the graft or repair.

In one preferred form of the invention, there is provided apparatus for extracting fibrin from blood so as to form fibrin biomaterials, the apparatus comprising:

a vessel for holding drawn blood;

a lid for selectively closing off the vessel; and

a precipitator connected to the vessel for engaging the drawn blood contained within the vessel and acting as a focal point for the precipitation of fibrin clot.

In another aspect of the invention, novel fibrin biomaterials having fibers are organized into largely parallel coherent strands or strings by forcing them through small openings.

In another aspect of the invention, fibrin is combined with cells or fillers and implanted into the body of a mammal.

### Processing System

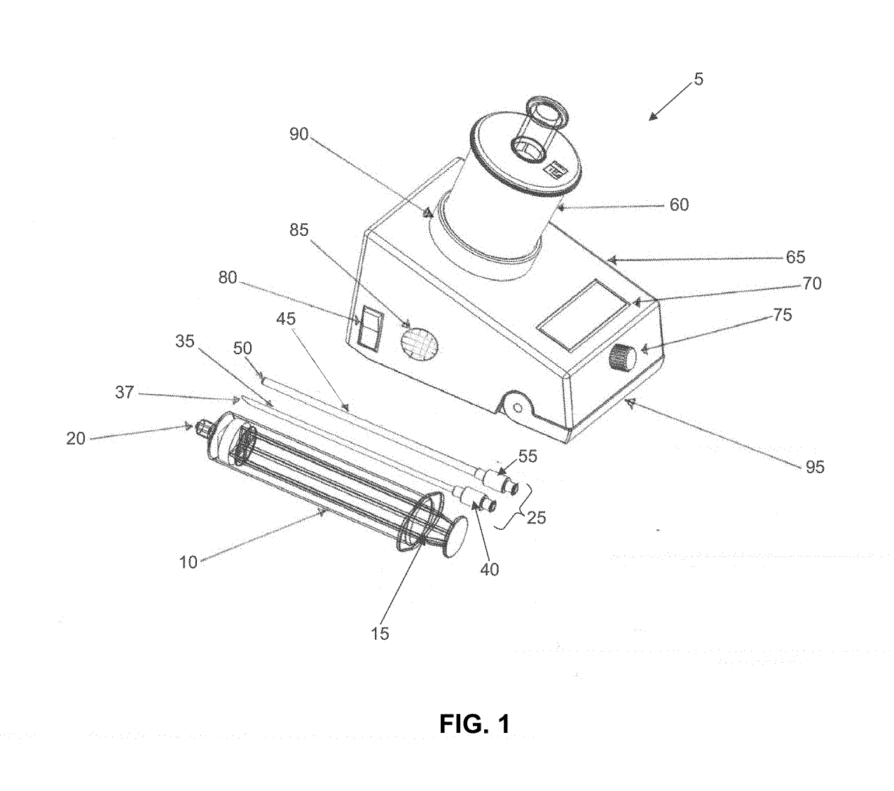

In one embodiment, and looking now at FIG. 1, system 5 comprises at least one syringe 10 having a syringe plunger 15 and a syringe opening 20 for drawing blood and fat; at least one liposuction needle combination 25 for aspirating fat from the body as well as separating congealed fibrin biomaterial, the liposuction needle combination 25 comprising a sharp injector needle 35 having a sharp or semi-sharp tip 37 for implanting biomaterial into the body and having a luer fitting 40 for a syringe, and a clot stripper 45 having a blunt tip 50 and a luer fitting 55 for a syringe; a vessel 60 for holding the drawn blood and additives (not shown) such as adipose cells and coagulating it together; and vessel agitator device 65 comprising a display 70, a speed controller 75, a power switch 80, a sonic indicator alarm or speaker 85, and a rotationally movable vessel receiving means 90 to induce fluidic motion within the vessel. Vessel agitator device 65 may also comprise a novel leveling armature 95.

For purposes of the present description, the contents of vessel 60 will hereinafter be referred to as drawn blood, however, it should be appreciated that the present invention is not intended to be limited to only drawn blood, but may also refer to drawn blood mixed with various additives, e.g., adipose-derived stem cells, collagen fibers, etc.

### Container Precipitator

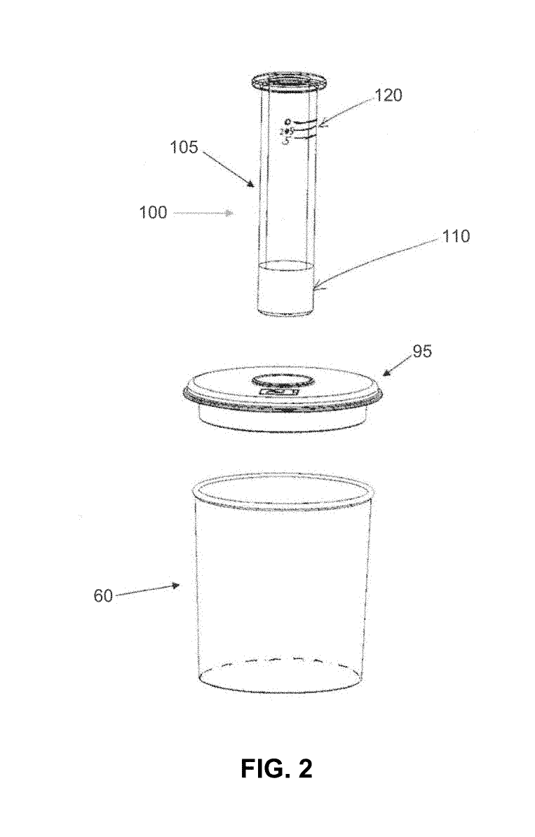

In one embodiment of the present invention, and looking now at FIGS. 2-4, system 5 comprises vessel 60 for holding drawn blood 92 (not shown), a lid 95 for selectively closing off vessel 60, and a precipitator 100 for precipitating fibrin clot from drawn blood 92 held in vessel 60 when drawn blood 92 is agitated vis-à-vis precipitator 100. In one preferred form of the invention, precipitator 100 comprises a rod 105 having an elongated shaft with a frosted glass section 110 adjacent to its distal end. Alternatively, precipitator 100 may comprise a plurality of rods, a paddle, a helical structure, a string of beads, a ball on a rod, a vane on a rod and/or any other appropriate configuration capable of precipitating fibrin clot from drawn blood 92 contained in vessel 60. For purposes of the present description, the present invention will hereinafter generally be discussed in the context of rod 105, however, it should be appreciated that the present invention is not intended to be limited to a precipitator having a rod construction.

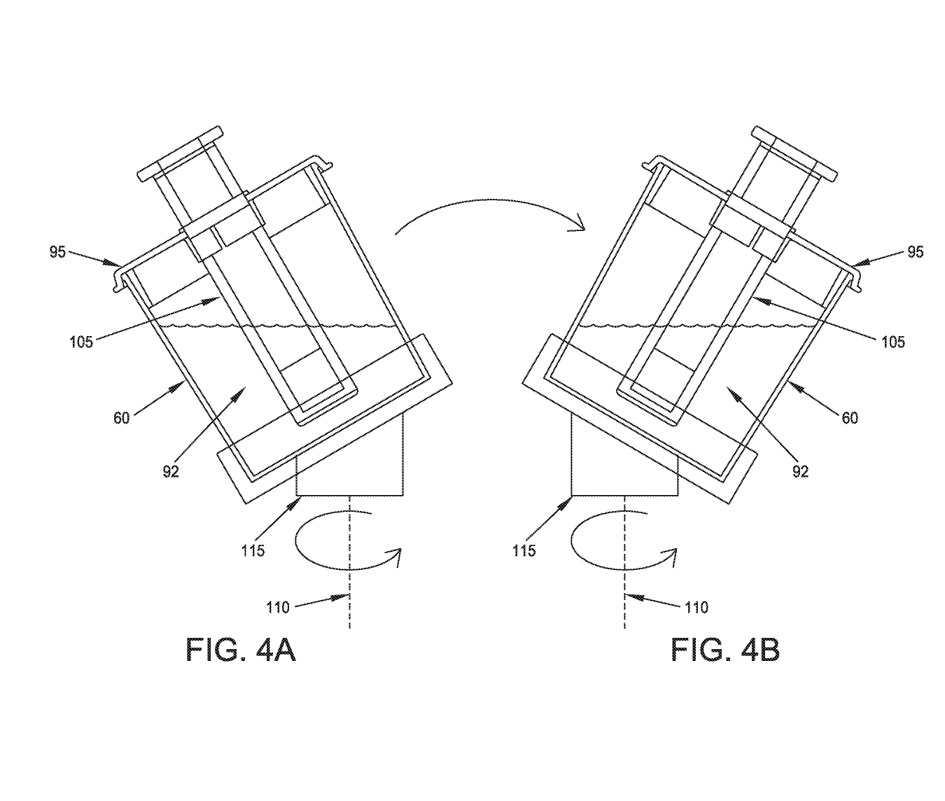

In one preferred embodiment of the invention, vessel 60 is agitated by tilting the vessel from side to side or by rotating the vessel about a center axis 110, or both (e.g., in the manner shown in FIG. 4, where vessel 60 is tilted and rotated using a motorized or mechanical tilter 115 which rotates about a center axis 110) so that drawn blood 92 contacts the surface of rod 105 and fibrin clot forms on rod 105. Thus it will be seen that rod 105 acts as a focal point for the precipitation of fibrin clot when drawn blood 92 in vessel 60 is agitated by tilting the vessel.

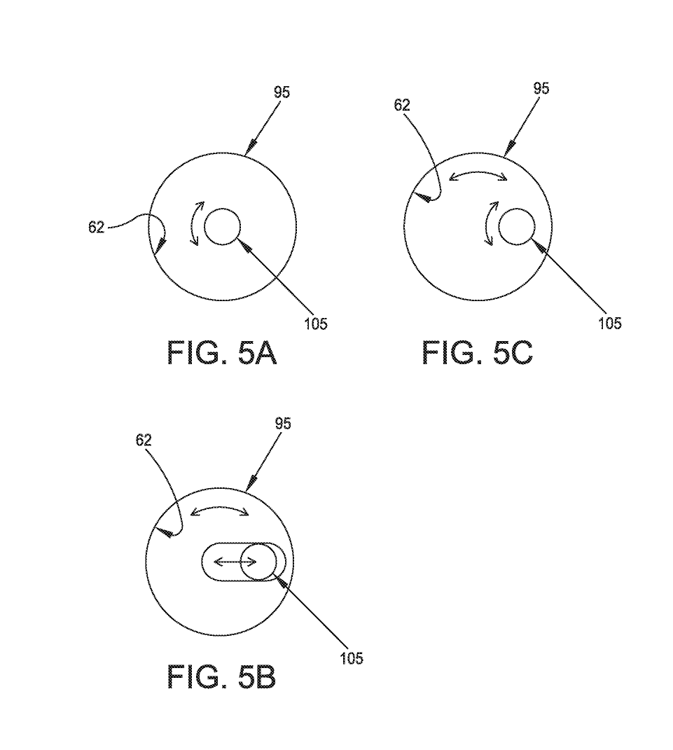

In another preferred embodiment of the present invention, and looking now at FIGS. 5A-5C the position of rod 105 can be altered relative to the side wall 62 of vessel 60, either for the purpose of agitating drawn blood 92 within vessel 60 or for inducing different fluid flow behaviors (and hence varied clot formation) within vessel 60 while vessel 60 is externally agitated, or both. Thus, for example, rod 105 may be rotatable relative to vessel 60 and lid 95 (FIG. 5A); and/or rod 105 may be radially movable relative to lid 95 (FIG. 5B); and/or rod 105 may be rotatable or fixed relative to lid 95, and lid 95 may be rotatable relative to vessel 60 (FIG. 5C), etc. Significantly, by being able to move the position of rod 105 relative to vessel 60, and particularly when vessel 60 is being externally agitated so as to induce blood flow within vessel 60, varied and optimized fluid flow can be induced so as to cause fibrin clot to aggregate on rod 105 and/or vessel walls with desired shapes and/or consistencies. Significantly, when rod 105 is spun on its axis while in close proximity to side wall 62 of vessel 60, fibrin clot of uniform thickness can be produced.

If desired, rod 105 may comprise line markings 120 (See FIG. 2) along its shaft so as to indicate to the user the distance which rod 105 extends into vessel 60. Alternatively, or in addition, vessel 60 can be formed with a transparent side wall 62 so that the user can observe the distance that rod 105 extends into vessel 60.

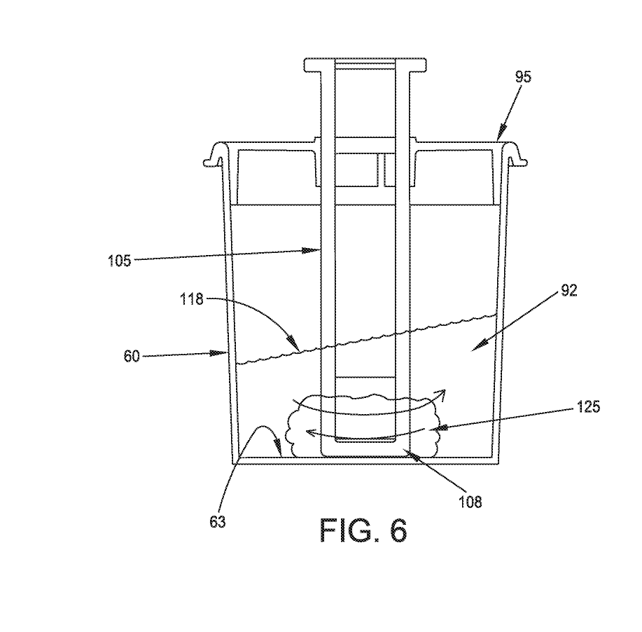

Significantly, and looking now at FIG. 6, it has been discovered that, by regulating the distance which rod 105 extends into the vessel—and, more specifically, by regulating the disposition of the distal end of rod 108 vis-à-vis the bottom surface 63 of vessel 60—a fibrin clot can be produced which is more uniform than the fibrin clot which can be produced by simply stirring a frosted glass rod in an open-topped bowl. This is a significant advance in the art, since a more uniform fibrin clot can be more easily handled by the user and more precisely inserted into the body.

More particularly, it has been discovered that the characteristics (e.g., shape, consistency, etc.) of the fibrin clot can be significantly influenced by the relative position of rod 105 vis-à-vis the bottom surface 63, or another internal surface, of the vessel.

Thus, and looking still at FIG. 6, where rod 105 is positioned against the bottom surface 63 of vessel 60, and vessel 60 is thereafter gently moved so as to cause drawn blood 92 to swirl circumferentially around the distal end of rod 105, the fibers of the formed fibrin clot tend to be oriented circumferentially around rod 105 in the direction of blood flow, and an annular (e.g., tubular, toroidal, etc.) fibrin clot 125 forms at the base of rod 105. This type of clot has particular utility as a breast implant because it will resist bursting or lateral deformation. The same annulus of clot can be cut to form a strip of parallel-oriented fibers that can be used to augment or replace a tendon, ligament, or soft tissue requiring support or suspensory tissue.

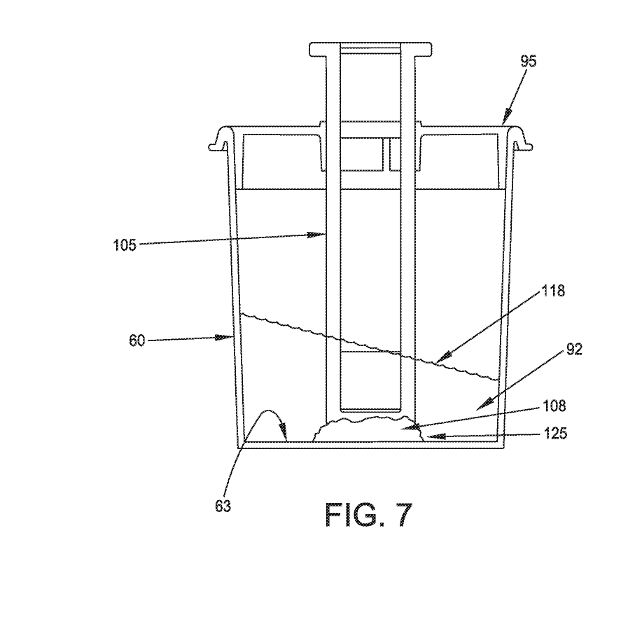

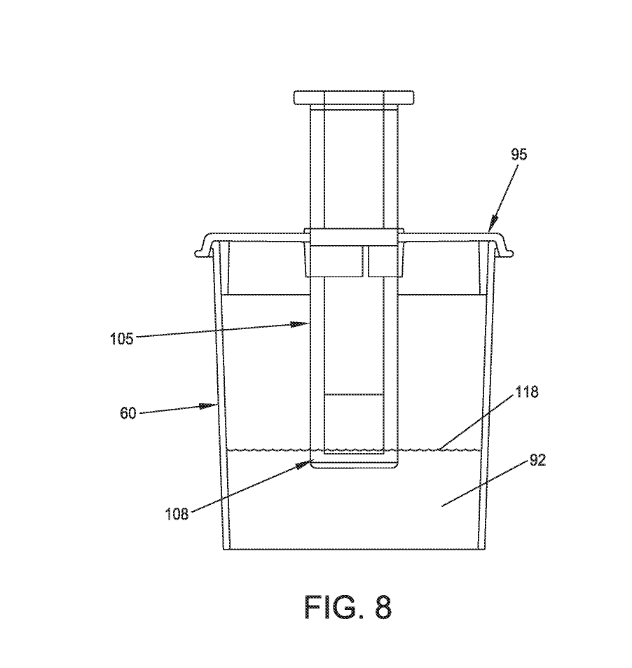

On the other hand, where distal end 108 of rod 105 is disposed so that it is spaced from the bottom surface of vessel 63, e.g., in the manner shown in FIG. 7, agitation of drawn blood 92 about rod 105 causes the fibrin clot 125 to take the shape of a flat, discoid membrane just beneath rod 105 (i.e., in the gap between the distal end 108 of rod 105 and the bottom surface 63 of vessel 60).

Where rod 105 is disposed so that its distal tip 108 is located just below the surface 118 of drawn blood 92, e.g., in the manner shown in FIG. 8, and where drawn blood 92 is thereafter gently tilt-stirred for approximately five minutes or less and then allowed to rest (i.e., stand) for approximately another five minutes, drawn blood 92 typically gels into a liver-like consistency, whereupon the gel can be handled without falling apart and the platelet-poor plasma can be extruded upon application, leaving an adhesive mat or infill of clot.

Preferably, rod 105 is formed out of a sintered glass rod. Rod 105 may also be formed of another material which is configured to precipitate fibrin clot, e.g., a metal member such as steel, or a non-glass material, or an open-ended tube that traps air, etc.

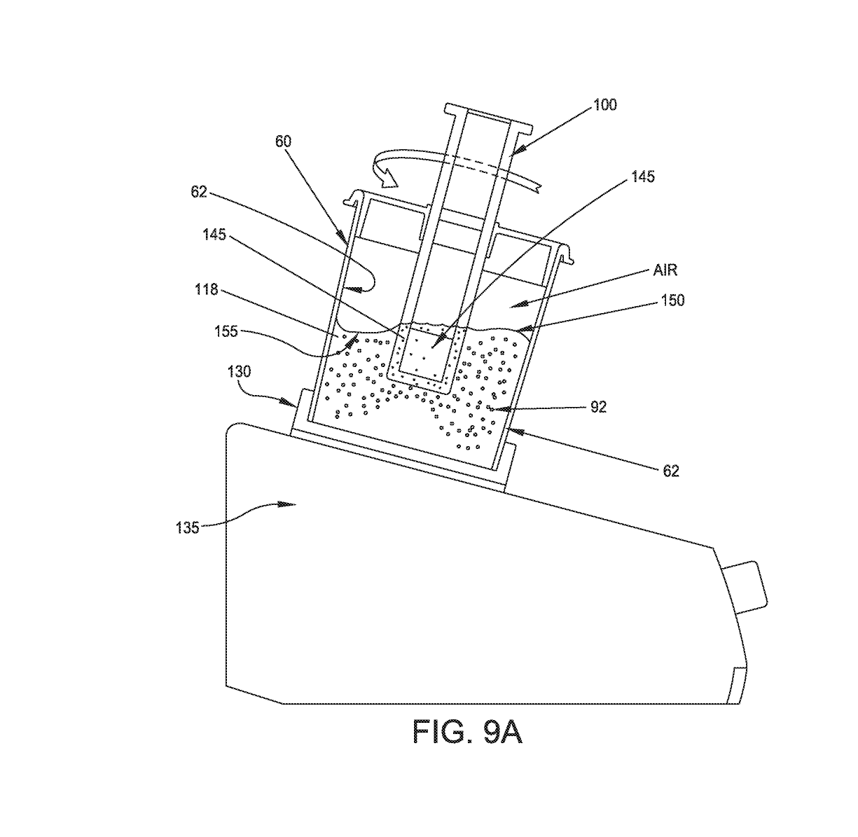

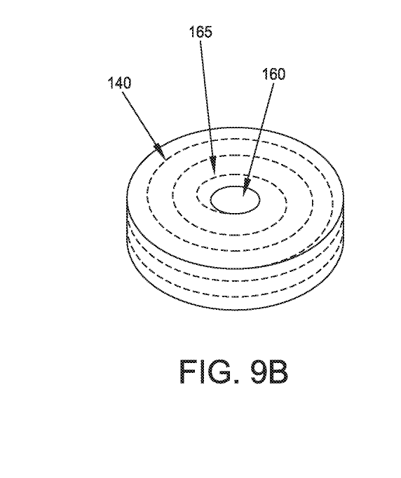

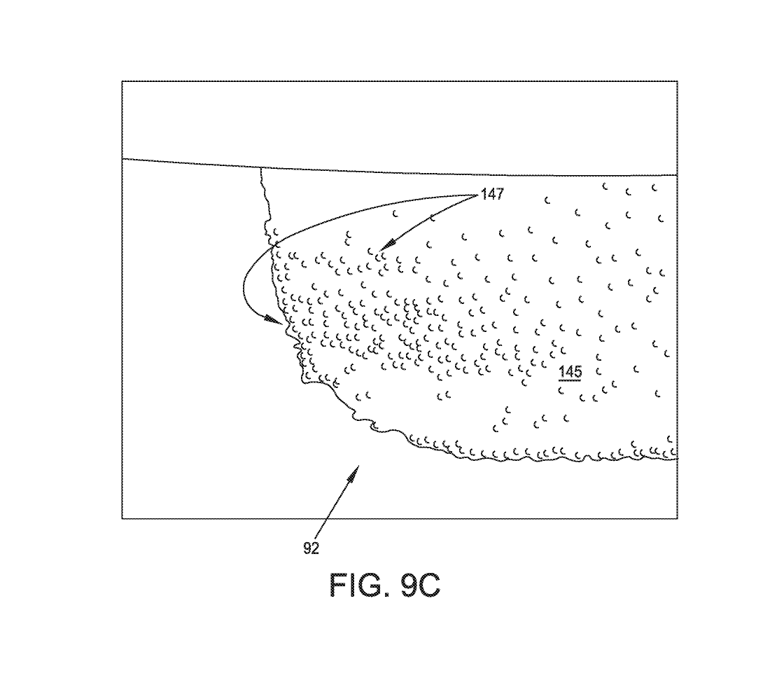

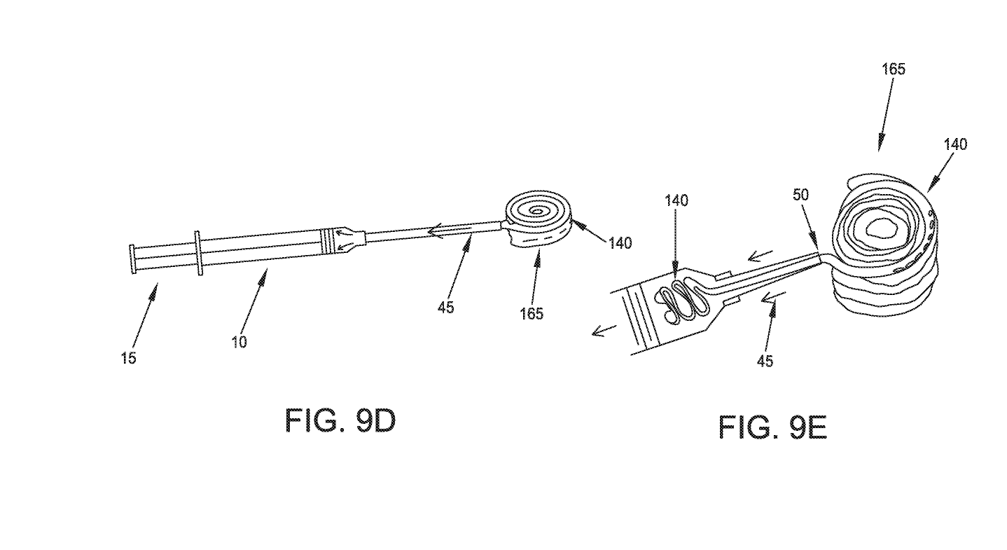

Looking now at FIG. 9A, a cross-sectional view of system 5 is shown, with vessel 60 on a rotating turntable 130 mounted to a motorized spinner base 135, where production of a fibrin biomaterial with circumferentially-oriented fibers 140, such as that shown in FIG. 9B, is desired. Constant rotation of vessel 60 at an angle, e.g., 15 degrees, induces motion in drawn blood 92 with respect to the inner walls 62 of vessel 60 as well as with respect to rod 105 (which may comprise sintered glass 145). Both the vessel inner walls 62 and the sintered glass 145 create adherent air bubbles 147 that react with drawn blood 92 to induce clotting when drawn blood 92 is in motion. See FIG. 9C.

Micro bubbles of air play a role in clotting. Air trapped in the submerged frosted or sintered glass 145 forms bubbles on the surface of the glass. These bubbles interact with fibrinogen to promote the contact activation (intrinsic) pathway, initiated by activation of the “contact factors” of plasma by bubble surface tension effects and turbulence. The tissue factor (extrinsic) pathway is later initiated by release of tissue factor (a specific cellular lipoprotein). This cascade enables coherent gels to propagate through drawn blood 92 in the container.

Looking at FIG. 9A, the liquid surface also interacts with the inner walls 62 of vessel 60 in a reciprocating upwelling 150 and receding 155 action that imparts stresses caused by surface tension. Fibrin biomaterials created in this manner form a cohesive gel clot (or implant) 165 (shown in FIG. 9B) in about 10 minutes. Cohesive gel clot 165 possesses distinct circumferentially-oriented fibers 140 (see FIG. 9B), as well as an indentation 160 where the precipitator 100 (which may comprise glass, sintered glass or another material) contacted the liquid. The utility of the specific fiber orientation will provide structural matrix architecture that will tend to resist lateral and gravitational forces that could otherwise burst or damage the implant or allow the augmented tissue to stretch and sag over a period of years. Central indentation 160 also provides a useful channel for tissue and vascular ingrowth into the implant 165, and can allow central space to provide a more natural shape to the nipple apex of the breast. Those skilled in the art of tissue engineering will appreciate that such molded indentations can also be formed by more complex shaped spacers to create voids and encourage the formation of blood vessels, organ vesicles, fluid transport channels and the like.

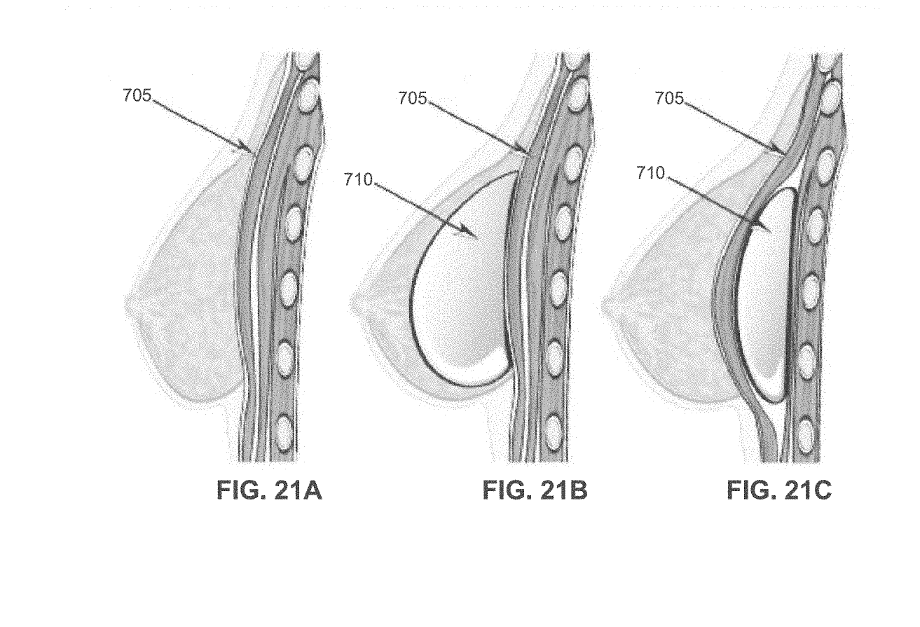

Looking now at FIGS. 9D and 9E, the fibrin implant material 165 comprising circumferentially-oriented fibers 140 can be processed into an injectable biomaterial by breaking it up and needle aspirating it into a syringe. The preferred means for doing this is by attaching a flush ended tube 45 to the lumen of syringe 10. Suction is applied to tube 45 and it is used to draw fibers with attached cells into the syringe body in a string form. This form can be re-injected also as a string or strands and will pile upon itself in a body cavity. This injectable form of the biomaterial invention is suitable for wrinkle repair as well as breast augmentation by submuscular or subglandular injection (see FIG. 21).

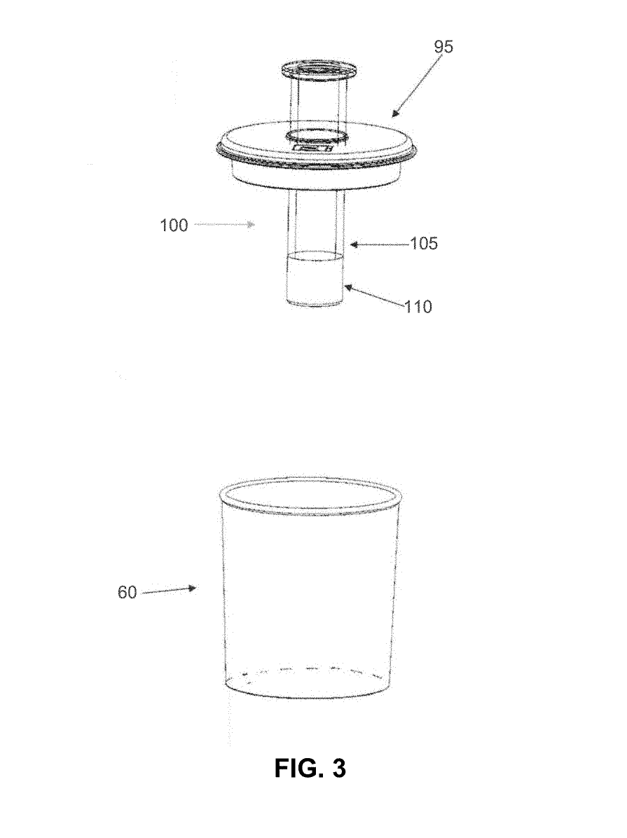

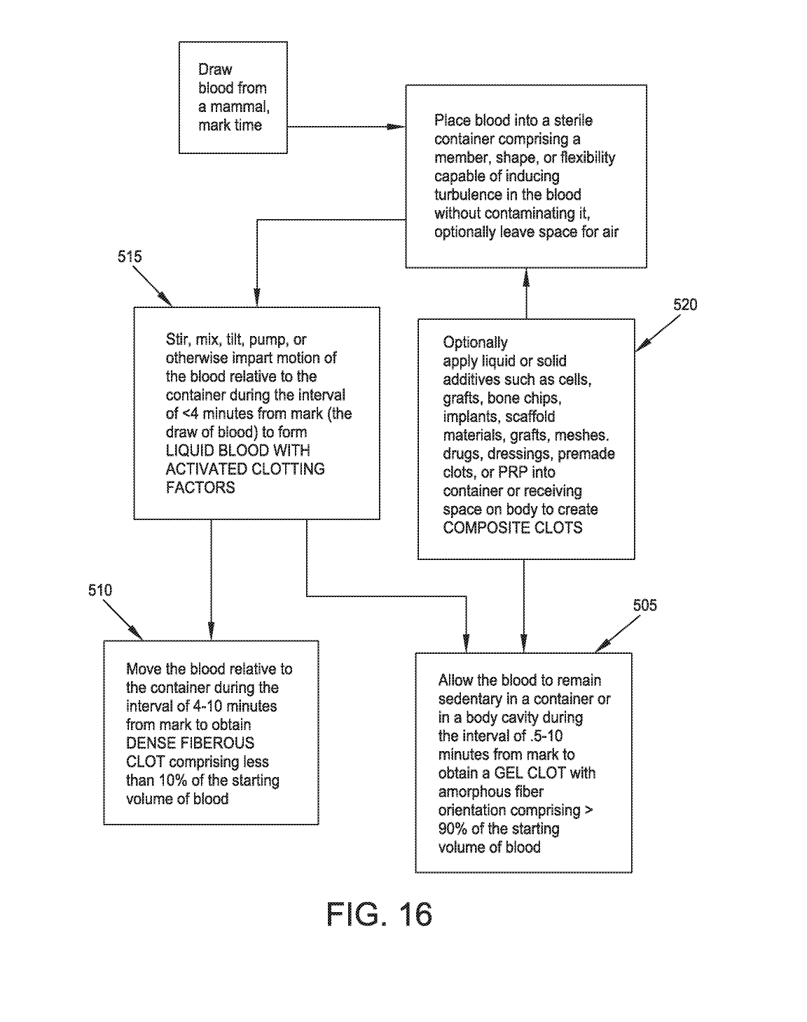

An alternate method of practicing the present invention comprises the steps of initiating the clotting cascade in vessel 60 shown in FIG. 3, by agitating the blood and any other contents for a period of time long enough to trigger the clotting cascade, (typically 0.5-3 minutes). The liquid biomaterial is then dispensed into a body cavity before the clotting cascade is completed (see FIG. 16) so that the biomaterial can set in-situ to form a lasting implant such as that shown in FIG. 21.

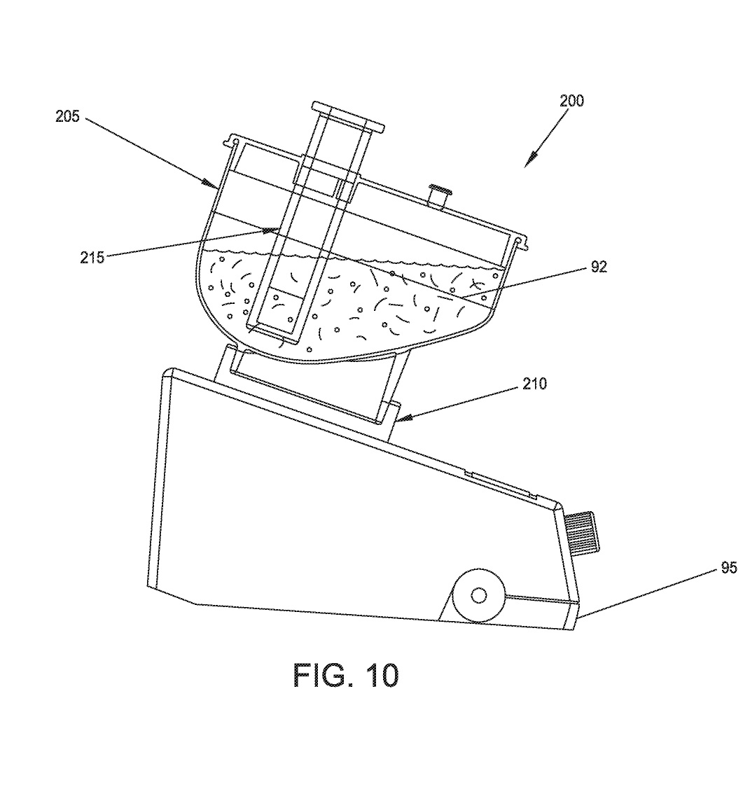

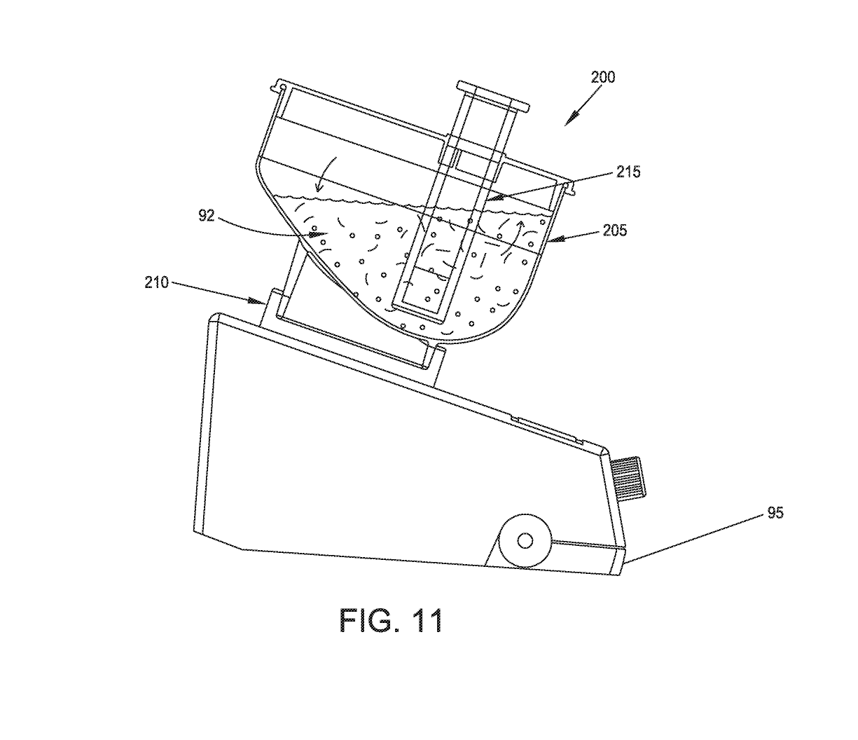

Looking now at FIGS. 10 and 11, there is shown an alternative embodiment 200 of the present invention. In the cross-sectional views shown in FIGS. 10 and 11, an asymmetrical vessel 205 (e.g., a breast implant mold) is shown that is rotated on an inclined turntable 210 at a rate of approximately 0.2-1.2 Hz for a period of 0.5-5.0 minutes to achieve mixing of blood and cells and initiation of the coagulation cascade as the blood and cells circulate around rod 215. Note that leveling armature 95 is not deployed.

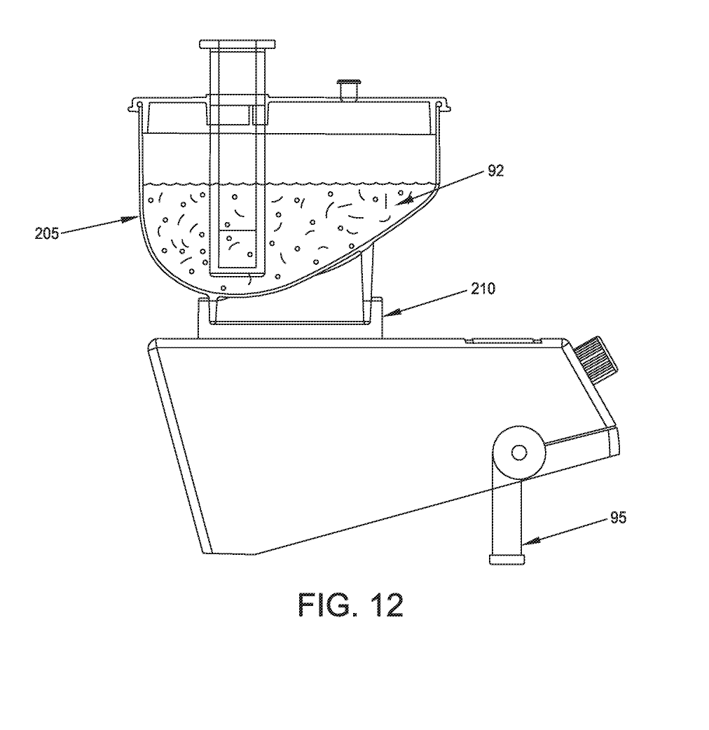

FIG. 11 shows a 180 degree rotated disposition of the asymmetrical vessel 205 during the vessel rotational cycle. When the turntable rotation of the device is stopped after typically 0.5-5.0 minutes, the leveling armature 95 is deployed to level the turntable and vessel/mold 205 (as shown in FIG. 12).

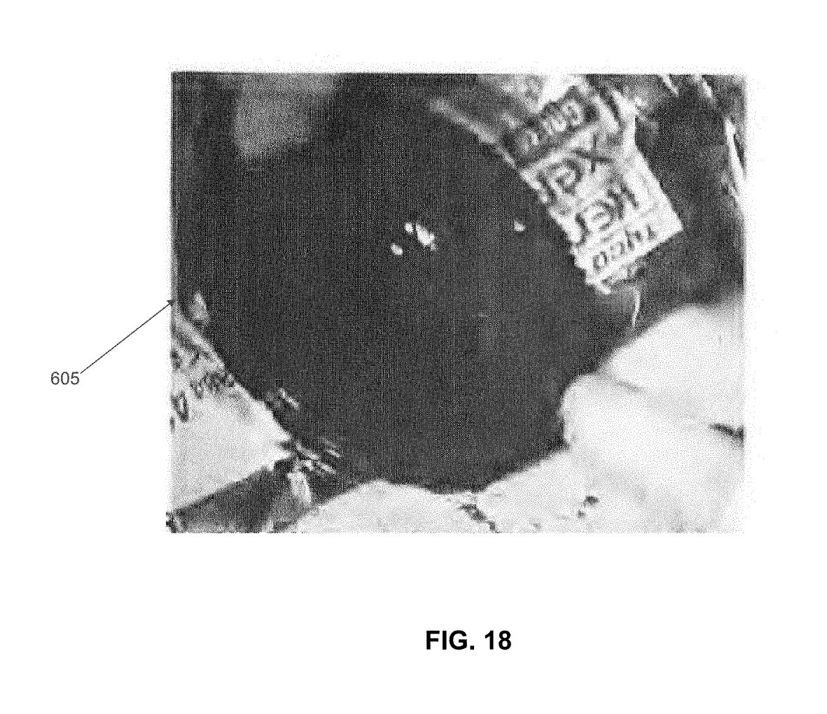

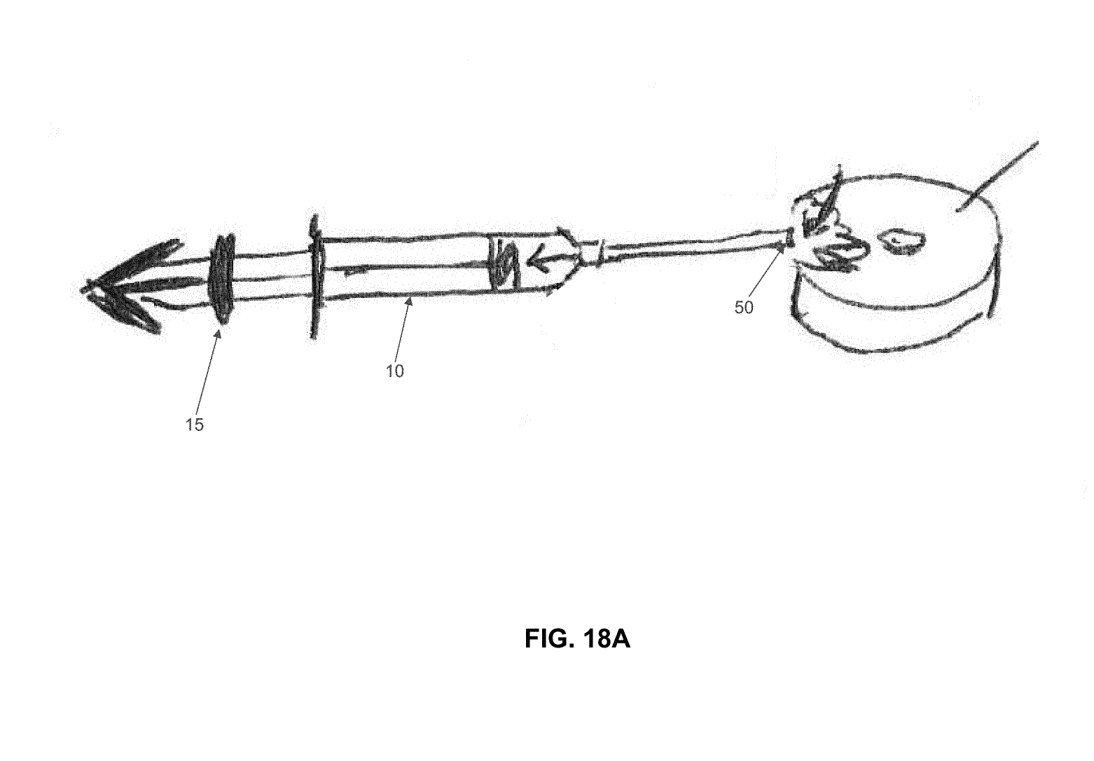

Looking now at FIG. 12, vessel/mold 205 and its contents are allowed to sit still (and level) for a period of at least 9-12 minutes to allow the clotting cascade to complete and form a molded implant having non-oriented cross-linked fibers that assumes the shape of vessel/mold 205 as shown in FIG. 18.

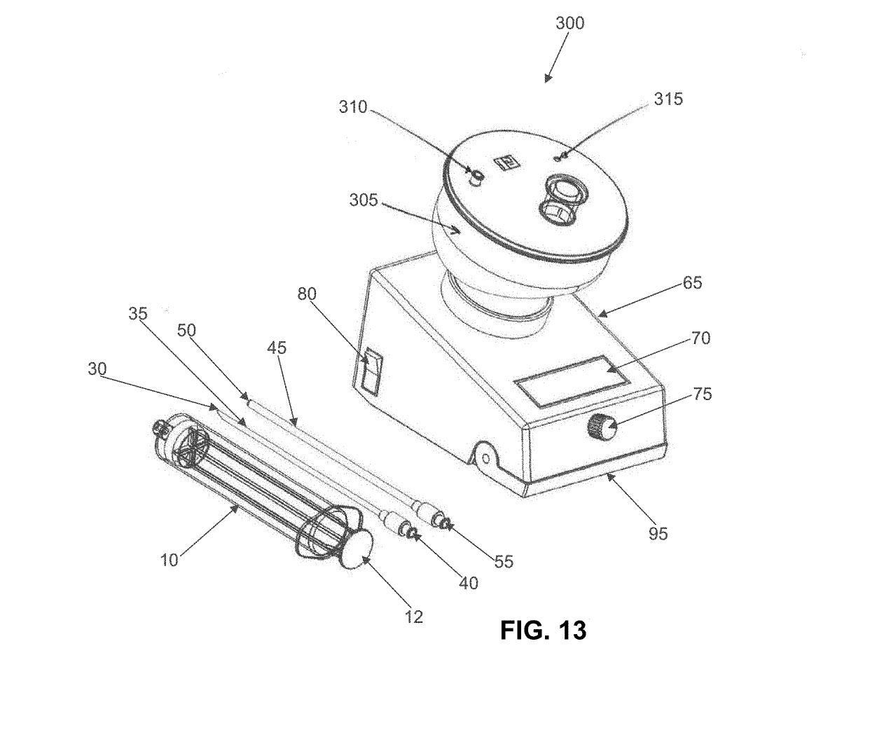

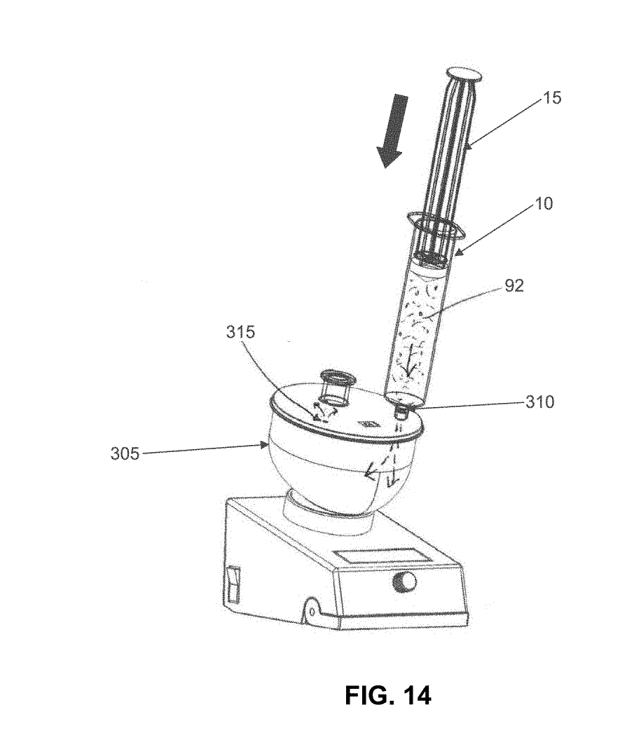

Looking now at FIGS. 13 and 14, another embodiment 300 of the present invention is shown. In FIGS. 13 and 14, a system 300 is shown for creating the molded implants described, where a breast implant mold 305 comprises a syringe port 310 for addition of liquids (e.g., drawn blood 92) and an air release port 315 to vent pressure upon filling.

FIG. 14 shows syringe 10 being attached to the syringe port 310 (input port) and plunger 15 being actuated so as to extrude liquids (e.g., drawn blood 92) inside of syringe 10 to fill vessel 305 with liquids.

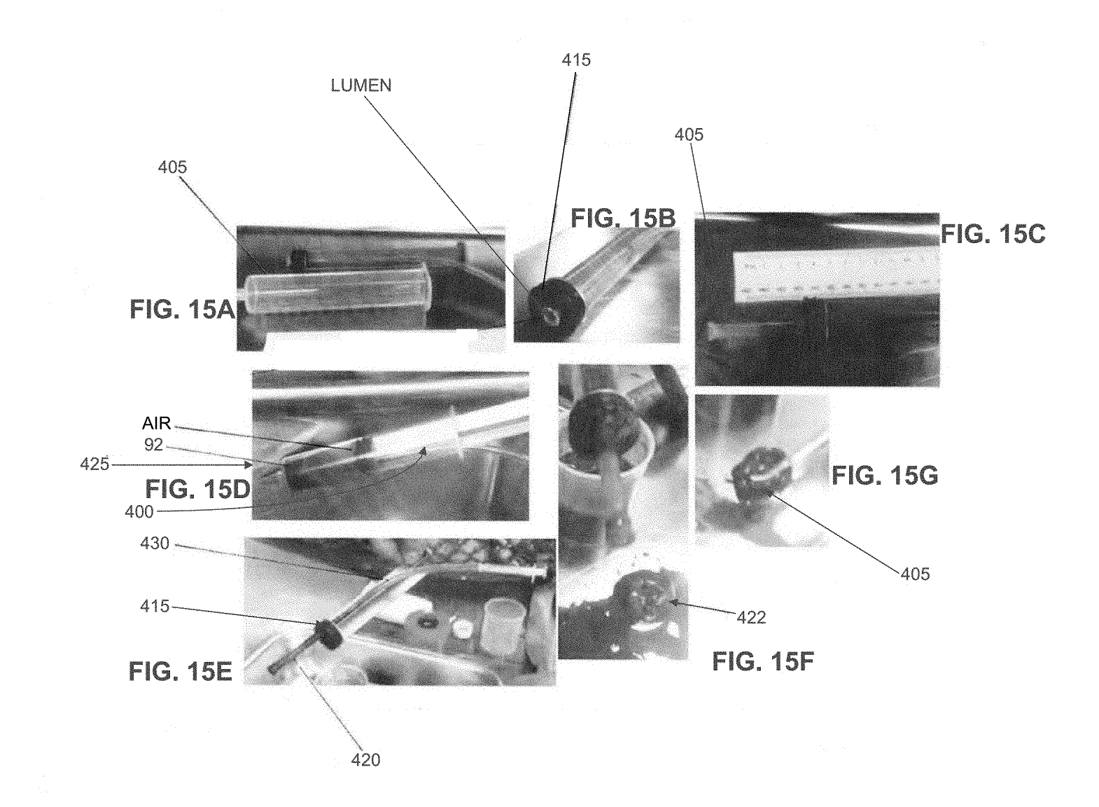

In another preferred form of the invention, and looking now at FIGS. 15A-15G, there is shown a syringe 405 for drawing blood or fat, a plunger 415 for movement along syringe 405, and a precipitator 420 which comprises a frosted glass tube along which the plunger 415 moves. This embodiment is capable of both drawing blood and clotting it. In this embodiment, high air-to-blood ratios enable denser clots to form. Gel clots will form in conditions of less fluidic mobility.

During or after blood is drawn into syringe 405 in the presence of air, a syringe nozzle 425 can be capped, or otherwise sealed, and the slidable precipitator tube 420 can be extended to contact the blood and initiate clotting to create a fibrin mass 422. The vent assembly 430 can comprise another syringe container or drain forming a fluid path communicating between the syringe interior and exterior and can be used by opening the vent and compressing syringe plunger 415 to vent off air and/or remove platelet poor plasma. In the case of fat processing, vent assembly 430 can be used to remove unwanted oil, lipids, and blood, or to enable exchange of air/fluid, bubble formation, suction/aspiration of clot, or addition of flowable materials, drugs, agents.

Alternate ways to use the device could involve separating fractions of blood or fat from either end of the inside of the syringe vessel that have been separated in the syringe vessel by centrifuge. For example, after a clot is formed (in contact with the precipitator), the vent in the precipitator, or in another portion of the plunger assembly can be opened, and the plunger can be advanced forcing platelet poor plasma red blood cells (RBCs) out of the syringe body and holding the clot in the main collection container (which may optionally also comprise a red blood cell filter).

FIG. 16 shows a flow chart delineating several optional ways the invention can be used to vary the type of biomaterial by hydration and fiber orientation. After application of agitation or agitation/sedation for approximately 10 minutes, a range of fibrin clot types, from gels having near baseline platelet counts and containing near or all of the platelet poor plasma (box 505), to dense fibrous clots that are platelet rich and possessing of much less platelet poor plasma (box 510) (the later having been separated) can be formed. The platelet poor plasma also contains red blood cells that can be removed by filtration or centrifugation, providing a medicament possessing a high content of desirable growth factors suitable for injection or reintroduction to tissue (box 515). The invention also provides a means to select the fiber orientation of monolithic or polymorphic fibrin clot scaffolds, enabling optimization of cell chemotaxis and microenvironmental cues to determine cell fate and integration into varied optimal tissue types.

Looking still at FIG. 16, a further object of the invention is to incorporate supplements into the scaffolds such cells, drugs, agents, hardeners, dressings, reinforcements, tissue grafts, and various hard and soft implants into clots (or vice versa) to form compound or composite biomaterials (box 520).

For example, the present invention can be used to lay down fibrin scaffold fibrils of selected orientations, such as parallel, concentric, or alternating layers as a means to form a breast implant that will resist sagging. Alternating layers can be formed by altering the direction of blood flow over the precipitation means. This can be achieved by subjecting the container to a sequence of varied motions to change the deposition induced alignment of fibril layers.

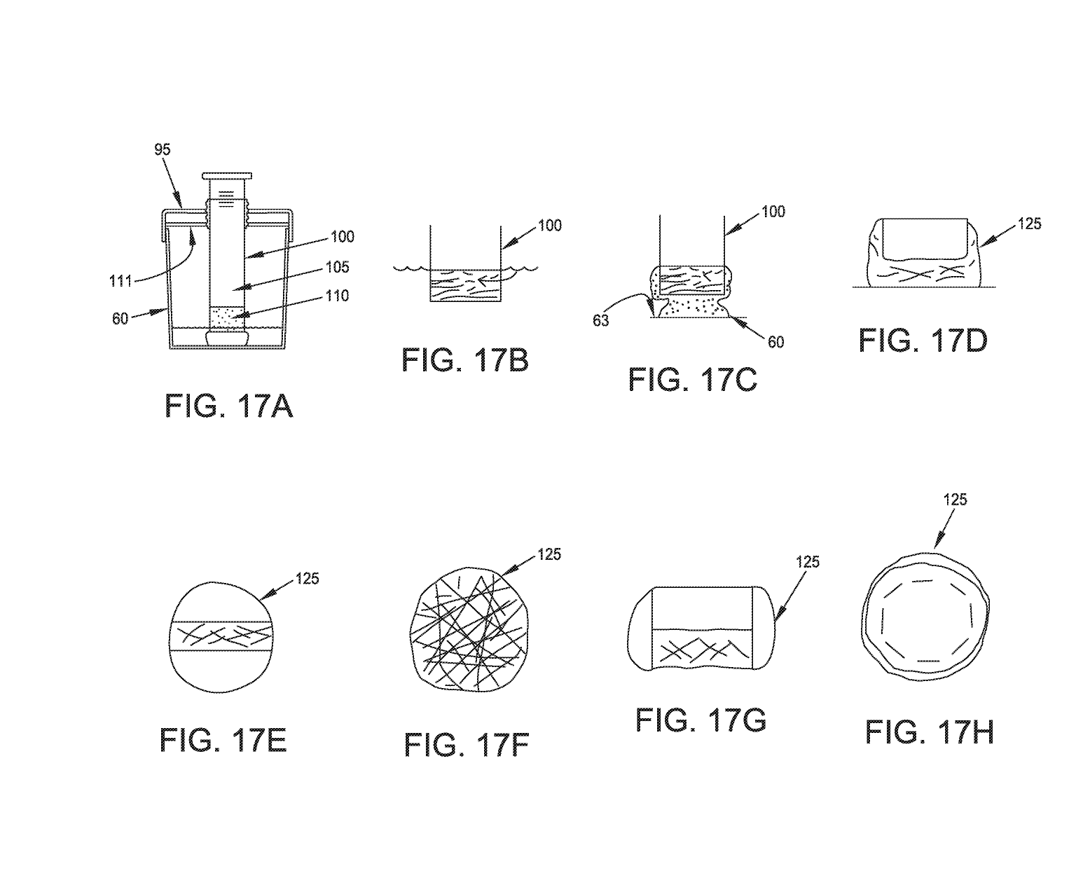

For example, and looking now at FIG. 17A, there is shown selected portions of the present invention including vessel 60, lid 95, precipitator 100, rod 105, and screen or absorbent material 111 to manage fluid and hold or catch clot after removing the clot from rod 105 and/or sintered glass 110.

Turning now to FIG. 17B, when blood and/or blood and other materials are moved relative to the precipitator 100 for approximately 5 minutes, clot will begin to form on the portion of precipitator 100 that is in contact with the blood and on the bottom surface of vessel 60.

After approximately 7 minutes of motion, a clot having multi-oriented fibrin will fill the gap between the bottom of precipitator 100 and the bottom surface 63 of vessel 60, while radially-oriented fibrin will comprise the portion of the clot formed around precipitator 100 as shown in FIG. 17C.

FIG. 17D shows a cross-section of clot 125 after 10 minutes of motion.

FIG. 17E shows that a flat discoid forms in the area of low volume flow in the area under the precipitator 100 and above the bottom surface 63 of vessel 60, and that the clot 125 molds to the vessel bottom (FIG. 17F).

FIG. 17G shows that radial fibers are formed last by flow around precipitator 100, and that discoid fiber orientation random in the x-y plane and largely oriented in the transverse axis are present in the clot 125.

The biomaterial of the present invention is unique because it enables the user to selectively create a diverse range of natural microenvironment structures, such as solid gels, flowable pastes, and dense fibrous forms having a particularly oriented or amorphous fiber structure. These biomaterials also provide unique benefits, e.g.: 1) the solid or gel fibrin maintains its architecture during implantation; 2) the matrix binds and delivers growth factors naturally over time; 3) fibrin biodegrades over a period of weeks and is replaced with derivative new tissue architecture; 4) the biomaterial allows seeded cells to persist with lower losses due to cell death; 5) the porosity allows motile phenotype healer cells to migrate into the open matrix; 6) the biomaterial is a regenerative mimic, pseudo-blastema, similar to the repair modality of erodeles and salamanders, where fibrin clots provide a scaffold for stem cells to regenerate missing or damaged tissue while minimizing or eliminating fibrosis; and 7) red blood cells, which could cause inflammatory response at the repair, adhere to the present biomaterial.

The present invention represents a significant and novel advance in biomaterials. Concentrated stem cells from marrow, fat, or culture methods can be incorporated into the various fibrin scaffold types by first positioning them in the container with non-anticoagulated blood or marrow, then inducing fluid motion within the device to activate the natural clotting cascade. In clinical practice, there is an attempt to mimic the target tissue fiber size and orientation of these fibrin scaffolds and augmentation materials, where possible.

Turning now to FIG. 18, there is shown Gel Clot Coherent Non-Oriented Cross-Linked Fibers 605, which are useable for repair augmentation, adhesion reduction, as a hemostat layer, or as a wound dressing.

In one aspect of the invention, blood is dispensed into the container and optionally agitated for up to 4 minutes to initiate the clotting cascade. Blood is allowed to sit with the glass rod or alternate precipitation means (such as air bubbles) touching the blood. The blood will gel and mold itself to the inside of the container taking on the shape of the mold. A small amount of platelet poor plasma will be expelled, only slightly reducing the volume of the clot gel from the original blood volume. The gel clot can also be formed upon various biomaterials, forming useful composites.

Utilizing the same apparatus described above, when agitation is minimized or ceased after a short period of stirring or agitation and the contents of the vessel 60 or syringe 405 are allowed to remain largely still, approximately 95-100% of the blood will form into a gel that assumes the shape of the container. This gel is coherent enough to be removed and handled, can be applied in sheets, blocks or segments, and is strong enough to be sutured into place. This gel looks and feels like liver and can be cut into useful shapes.

This coherent gel can be molded into varied shapes as it assumes the shape of the container that it forms in. For example, breast implants can be molded to appropriate shapes comprising aliquots of adipose cells. These gel clot constructs can also be broken up, shredded, pulled through a sieve or lumen (such as syringe 10 or blunt tip needle opening 50 shown in FIG. 1) or likewise morselized so that the materials can be injected through needles (such as a 21 gauge needle) and applied with a hypodermic syringe. See generally, FIG. 18A.

The gel will extravisate platelet poor plasma in handling, or when subjected to manual or physiologic point-load pressure, leaving denser fibrin and concomitant growth factors in-situ. The liquid platelet poor plasma trapped in the gels and expelled into the tissue with the implant is also rich in growth factors and serves as a biocompatible liquid delivery medium.

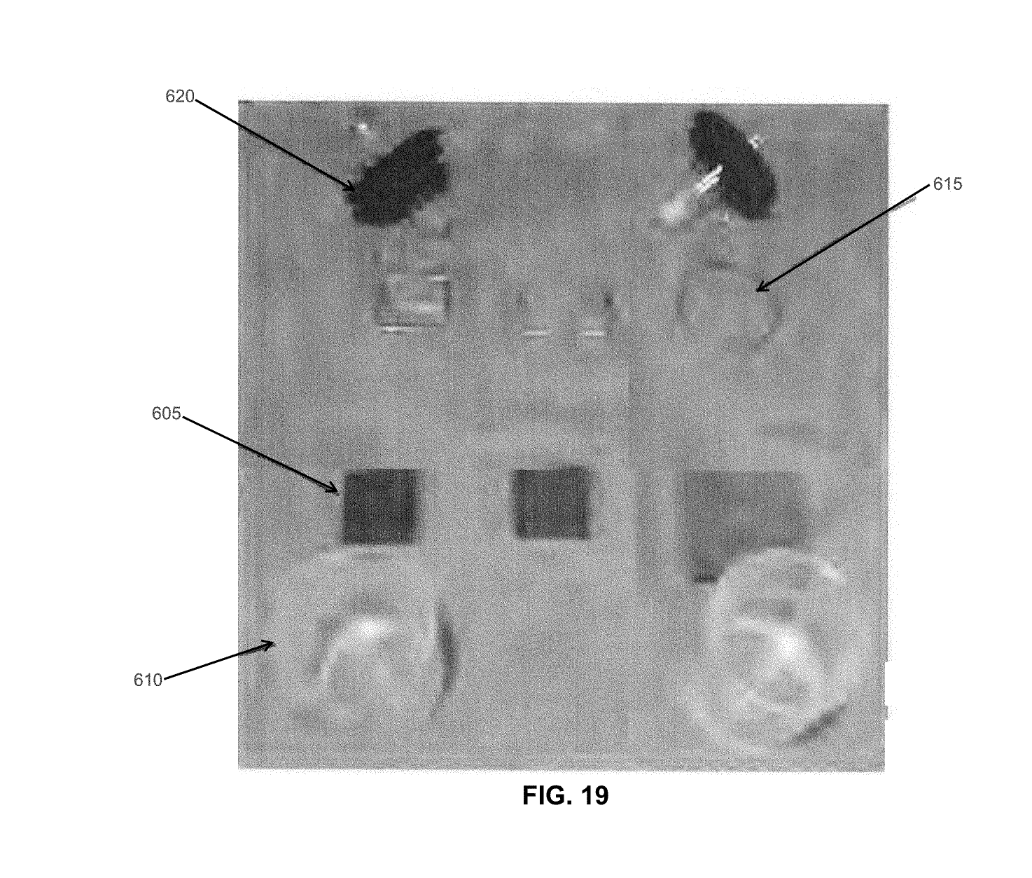

Looking now at FIG. 19, composite biomaterials 605 can be formed by positioning them in vessel 610 and forming clot onto, around, and within them. Implants such as xenografts, autografts, allografts, synthetics, textiles, sutures, straps, permanent or bioabsorbable structural materials can be positioned within vessel 610 and can be confined by various holders 615. Free-floating materials such as bone void fillers, collagen fibers, drug bearing granules, cells, tissue, growth factors, genetic material (such as plasmid DNA, etc.) can be added and entrapped in the clot to provide immediate structural integrity or long term tissue for repair or regeneration of a desired tissue type. A clot with the added and entrapped materials can be used for later implantation, or topical application.

Looking still at FIG. 19, sedentary cycles create gels and these gels penetrate other biomaterials. Flowable or unconfined materials, fibers, scaffolds, drugs, cells or agents can be added to the blood and will be captured in the composite. Also referring to FIG. 19, various biologic or synthetic tissue reinforcement implants or dressings are also shown as holders and positioning means to orient and fixate the implant in the vessel/mold 610. The holders may be used to hold tubular materials open or to keep flat materials positioned on the base of the container. These holders can easily be connected to the lid 620 or the interior of the vessel.

An alternative use for this gel clot is to form it in a container directly onto a soft tissue reinforcing implant where growth factor release and intimate fibril formation on the implant surface is desired to promote integration of bone or soft tissue.

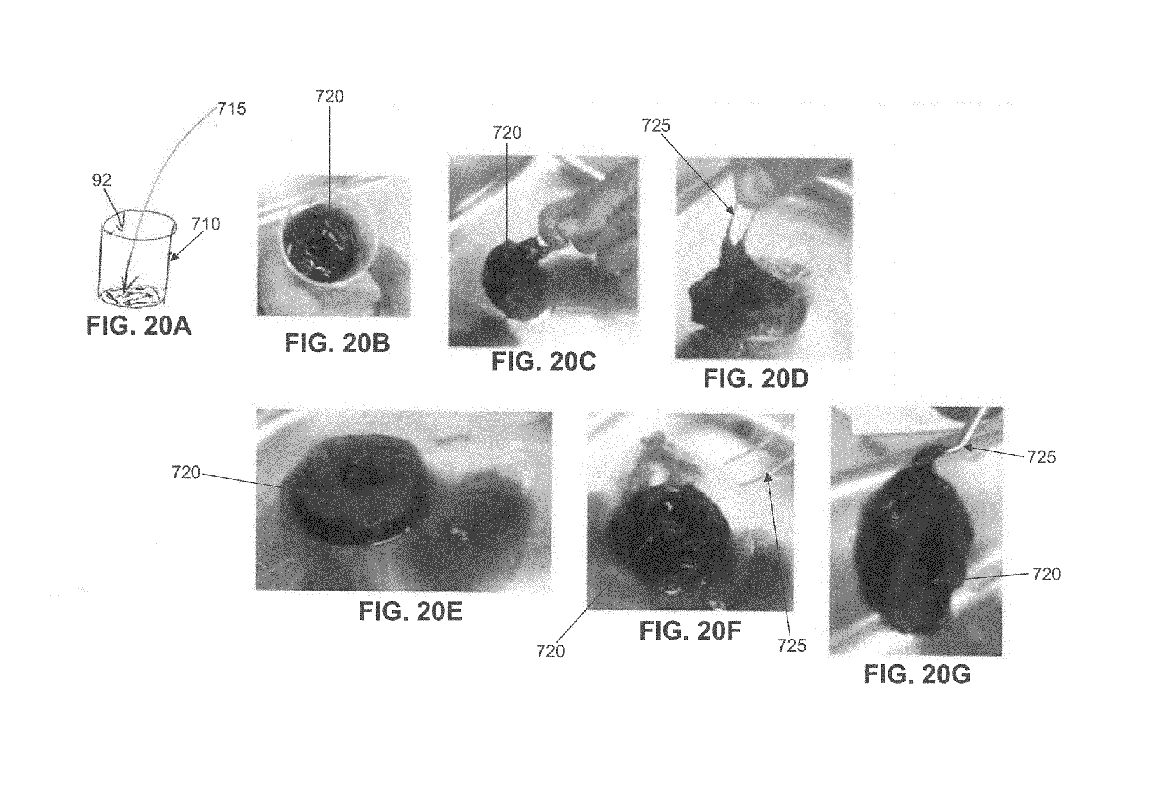

Looking now at FIGS. 20A-20G, blood and cells 92 can be added to a vessel 710 containing a textile or other implant 715, and subjected to gentle swirling for 1 minute and then allowed to stand for another 9 minutes. The blood mixture gels on the textile and assumes the shape of the container, forming a composite implant 720. The use of this technique with molds having intricate shapes will be evident to those skilled in the art.

Looking now at FIGS. 19-20G, various static and mobile positioning devices can be used in practice to target the integration and formation of different clot types with different bioabsorbable material grafts and implants. Handles 725 can be used to extract the composite, as shown in FIGS. 20C, 20D and 20G. Holder 725 can be pulled free of the composite implant. The textile holder may also be pulled free of the composite disk. The implant (composite disk) is then ready to be applied to the body.

Further, a method and apparatus of forming implants where liquid blood is subjected to motion and mixing to initiate a clotting cascade, and immediately applied to a repair site (such as shown in FIG. 21) in liquid form where it is then allowed to form a cohesive infilling clot in the body is also taught herein. Said cohesive infilling clot is formed in approximately 10 minutes from the initiation of mixing.

For example, and looking still at FIG. 21, a cohesive circumferential fibrin surgical implant 710 (optionally comprising cells) is shown and can be implanted submuscularly (behind pectoral muscle 705) or subglandularly (in front of pectoral muscle 705) like conventional breast implants. The adhesive nature of fibrin serves to retain the shape of the implant, and the rapid ingrowth of surrounding tissue further serves to stabilize the implant. An alternative method is to initiate clotting for less than 4 minutes and inject the liquid into the subglandular or subpectoral space, where it will form a gel in approximately 10 minutes.

The present invention offers a significant advance in the art of cosmetic and plastic surgery by providing a means to entrap adipocytes in a fibrin delivery material that can be formed into semi-solid gels or shapes, or subsequently broken up to be injected as a stranded liquid bearing fibrin fibrils mixed with fat cells.

### Additional Aspects of the Present Invention

In one aspect of the present invention, there is provided an apparatus for extracting fibrin from blood so as to form a fibrin biomaterial, the apparatus comprising:

a vessel for holding drawn blood and additives such as cells;

a lid, cap, plunger or closure means for selectively sealing the vessel;

a precipitator connected to the lid for engaging the drawn blood contained within the vessel and acting as a focal point for the precipitation of fibrin clot; and

a means for imparting fluid motion relative to the container.

In one aspect, the precipitator comprises a rod.

In one aspect, the rod is movably connected to the lid.

In one aspect, the rod is rotatable relative to the lid.

In one aspect, the rod is radially slidable along to the lid.

In one aspect, the rod is movable longitudinally relative to the lid.

In one aspect, the lid is rotatable relative to the vessel.

In one aspect, the distal end of the rod engages the bottom surface of the vessel.

In one aspect, the distal end of the rod is spaced a small distance from the bottom surface of the vessel.

In one aspect, the distal end of the rod is disposed just below the top surface of the drawn blood contained within the vessel.

In one aspect, the rod comprises line markings.

In one aspect, the vessel is transparent and comprises volume markings.

In one aspect, the rod comprises sintered glass that forms and holds air bubbles when immersed in liquid.

In one aspect, the vessel is a syringe for drawing blood, the lid is a plunger movable along the syringe, and the precipitator is a rod along which the plunger moves.

In one aspect, the vessel comprises a mold for shaping a fibrin biomaterial implant.

In one aspect, the means for imparting fluid motion relative to the container comprises a motor and a receiving means for the vessel.

In one aspect, the fluidic motion generator comprises a timer and alert means for selection of a cycle of motion more than 30 seconds, and less than 6 minutes followed by a period of sedation of at least 4 minutes for a cohesive gel biomaterial to form.

In one aspect, the fluidic motion generator comprises a timer for selection of a 6-15 minute cycle of motion for a cohesive gel biomaterial having circumferential fiber orientation to form.

In another aspect of the present invention, there is provided a method for forming fibrin biomaterial, the method comprising:

placing drawn blood and fillers such as live cells in a vessel;

mounting a precipitator to the vessel so that the precipitator extends into the material contained in the vessel; and

agitating the drawn materials so as to cause fibrin clot to form that is directly bonded to the filler materials.

In yet another aspect, the fibrin biomaterial is placed into a vessel or tubing and extruded through a small opening so as to entwine and form parallel-oriented fibrin fibrils into a yarn or string like material into a body cavity.

In yet another aspect, the fibrin biomaterial is extruded through successively smaller openings so it can be dispensed through a small applicator without clogging it.

In yet another aspect of the present invention, there is provided an apparatus for extracting fibrin from blood so as to form a fibrin clot optionally combined with living cells, the apparatus comprising:

a vessel for holding drawn blood and cells;

a lid for selectively closing off the vessel;

a precipitator connected to the lid for engaging the drawn blood contained within the vessel and acting as a focal point for the precipitation of fibrin clot; and

a means to manually or mechanically inducing fluidic motion within a container to elicit selectively variable matrixes as tissue fillers and cell delivery implants and biomaterials.

### Modifications

It will be understood that many changes in the details, materials, steps and arrangements of parts, which have been herein described and illustrated in order to explain the nature of the invention, may be made by those skilled in the art without departing from the principles and scope of the present invention.

What is claimed is:

1. A composite biomaterial comprising at least one blood component and adipose-derived stem cells wherein the at least one blood component is bonded to the adipose-derived stem cells as the at least one blood component coagulates.

2. A composite biomaterial according to claim 1 wherein the at least one blood component comprises fibrin.

3. A composite biomaterial according to claim 1 wherein the at least one blood component comprises growth factors, and further wherein the concentration of growth factors is greater at the periphery of the composite biomaterial.

4. A composite biomaterial according to claim 1 wherein the composite biomaterial comprises parallel-oriented fibers.

5. A composite biomaterial according to claim 1 wherein the composite biomaterial comprises a gel-like clot with non-oriented cross-linked fibers.

6. A method for making a composite biomaterial comprising:
extracting at least one blood component from a body;
extracting adipose-derived stem cells from a body;
placing the at least one blood component and adipose-derived stem cells in a vessel; and
bonding the at least one blood component to the adipose-derived stem cells as the at least one blood component coagulates.

7. A method according to claim 6 wherein the vessel is moved so as to provide relative motion between the vessel and the at least one blood component and adipose-derived stem cells.

8. A method according to claim 7 wherein the vessel is moved by a motor.

9. A method according to claim 6 wherein the vessel is moved at an angle to the vertical axis.

10. A method according to claim 9 wherein the vessel is moved so as to create a reciprocating upwelling and receding of the at least one blood component and adipose-derived stem cells with respect to the walls of the vessel.

11. A method according to claim 6 wherein the vessel is moved so as to create air bubbles in the vessel.

12. A method according to claim 6 wherein the vessel is moved so as to create a composite biomaterial with parallel-oriented fibers.

13. A method according to claim 6 wherein the vessel is moved so as to create a composite biomaterial with a gel-like consistency.

14. A method according to claim 6 wherein the composite biomaterial comprises a mass with an indentation.

15. A method for treating a patient, the method comprising:
extracting at least one blood component from a body;
extracting adipose-derived stem cells from a body;
placing the at least one blood component and adipose-derived stem cells in a vessel;
activating at least one blood component so that at least one blood component binds to the adipose-derived stem cells as the at least one blood component coagulates; and
deploying the composite biomaterial into a human body.

16. A method according to claim 15 wherein the composite biomaterial is deployed into a human body using a syringe.

17. A method according to claim 16 wherein the composite biomaterial is extruded through successively smaller diameter openings provided in passageways connected to the syringe.

18. A method according to claim 15 wherein the composite biomaterial is deployed into a human body as a pre-formed implant.

19. Apparatus for making a composite biomaterial, the apparatus comprising:
a vessel for holding liquids;
a lid for selectively closing off the vessel; and
a precipitator.

20. Apparatus according to claim 19 wherein the apparatus further comprises a motorized turntable for moving the vessel.

21. Apparatus according to claim 20 wherein the motorized turntable comprises a leveling armature.

22. Apparatus according to claim 21 wherein the precipitator comprises a frosted or sintered glass rod.

23. Apparatus according to claim 19 wherein the precipitator is movable relative to the walls of the vessel.

24. Apparatus according to claim 19 wherein the precipitator is movable relative to the lid.

25. Apparatus according to claim 19 wherein the vessel comprises a syringe.

26. Apparatus according to claim 19 wherein the lid comprises a plunger.

27. Apparatus according to claim 19 wherein the vessel comprises a mold for forming an implant.

28. A method for treating a patient, the method comprising:
extracting at least one blood component from a body;
extracting adipose-derived stem cells from a body;
placing the at least one blood component in a vessel;
clotting the at least one blood component in a vessel;
mixing the adipose-derived stem cells with the at least one clotted blood component to form a composite biomaterial; and
deploying the composite biomaterial into a human body.
